(19) United States
(12) Patent Application Publication
(10) Pub. No.: US 2024/0024877 A1
(43) Pub. Date: Jan. 25, 2024

(54) MICROFLUIDIC DEVICE AND CLASSIFICATION METHOD

(71) Applicants: CANON MEDICAL SYSTEMS CORPORATION, Tochigi (JP); Canon Kabushiki Kaisha, Tokyo (JP)

(72) Inventor: Yosuke YAMAGUCHI, Kawasaki (JP)

(73) Assignees: CANON MEDICAL SYSTEMS CORPORATION, Tochigi (JP); Canon Kabushiki Kaisha, Tokyo (JP)

(21) Appl. No.: 18/358,539

(22) Filed: Jul. 25, 2023

(30) Foreign Application Priority Data
Jul. 25, 2022 (JP) 2022-117820

(51) Int. Cl. B01L 3/00

(52) U.S. Cl. CPC B01L 3/502761; B01L 2200/0652; B01L 2300/088

(57) ABSTRACT

A microfluidic device according to an embodiment includes a microchannel and an inflow part. The microchannel is configured to separate particles contained in a fluid at least in a first direction according to the size of the particles by the action of lift force, and separate the particles in a second direction by the action of flow in a channel cross-section. The inflow part is provided on the upstream from an area where the lift force acts in the microchannel and allows the fluid to flow into the area where the lift force acts. A length of a channel cross-section of the inflow part in the first direction is formed to be smaller than a length of a channel cross-section of the area where the lift force acts in the first direction.

### CROSS-REFERENCE TO RELATED APPLICATIONS

This application is based upon and claims the benefit of priority from Japanese Patent Application No. 2022-117820, filed on Jul. 25, 2022; the entire contents of which are incorporated herein by reference.

### FIELD

Embodiments described herein relate generally to a microfluidic device and a classification method.

### BACKGROUND

In order to establish induced pluripotent stem (iPS) cells, processing of classifying nucleated cells from blood is necessary. Here, centrifugation is known as a common technique to classify nucleated cells from blood. However, there is a concern that in centrifugal classification, nucleated cells may be damaged due to the large centrifugal force applied to the nucleated cells, and there is a demand for a classification method that causes less damage to the nucleated cells.

A method of using a microchannel is known as a classification method that is less damaging to nucleated cells than centrifugation. This method is a method of separating particles having various sizes in a channel cross-section by advection due to the Dean vortex generated in a helical microchannel and the lift force caused by the velocity distribution on a channel wall.

### BRIEF DESCRIPTION OF THE DRAWINGS

FIG. 1 is a block diagram illustrating an example of a configuration of a microfluidic device according to a first embodiment;

FIG. 2 is a diagram for explaining a microchannel that classifies particles contained in a fluid by the action of lift force and Dean vortex;

FIG. 3A is a diagram illustrating an example of an inflow part according to the first embodiment;

FIG. 3B is a diagram illustrating an example of the inflow part according to the first embodiment;

FIG. 3C is a diagram illustrating an example of the inflow part according to the first embodiment;

FIG. 4A is a diagram for explaining a result of a simulation according to the first embodiment;

FIG. 4B is a diagram for explaining the result of the simulation according to the first embodiment;

FIG. 5A is a diagram for explaining an example of an inflow part according to another embodiment;

FIG. 5B is a diagram for explaining an example of shapes of the inflow part and a microchannel according to the other embodiment;

FIG. 5C is a diagram illustrating an example of a shape of the inflow part according to the other embodiment;

FIG. 6A is a diagram illustrating an example of a shape of an inflow part according to another embodiment;

FIG. 6B is a diagram illustrating the example of the shape of the inflow part according to the other embodiment;

FIG. 6C is a diagram illustrating the example of the shape of the inflow part according to the other embodiment; and

FIG. 7 is a diagram illustrating an example of a shape of an inflow part according to still yet another embodiment.

### DETAILED DESCRIPTION

A microfluidic device according to an embodiment includes a microchannel and an inflow part. The microchannel is configured to separate particles contained in a fluid at least in a first direction according to the size of the particles by the action of lift force, and separate the particles in a second direction by the action of flow in a channel cross-section. The inflow part is provided on the upstream from an area where the lift force acts in the microchannel and allows the fluid to flow into the area where the lift force acts. A length of a channel cross-section of the inflow part in the first direction is formed to be smaller than a length of a channel cross-section of the area where the lift force acts in the first direction.

The embodiment of the microfluidic device and a classification method will be described in detail below with reference to the accompanying drawings. The microfluidic device and classification method according to the present application are not limited to the following embodiments. In the following description, the same components are given a common numeral and duplicated explanations are not repeated.

### First Embodiment

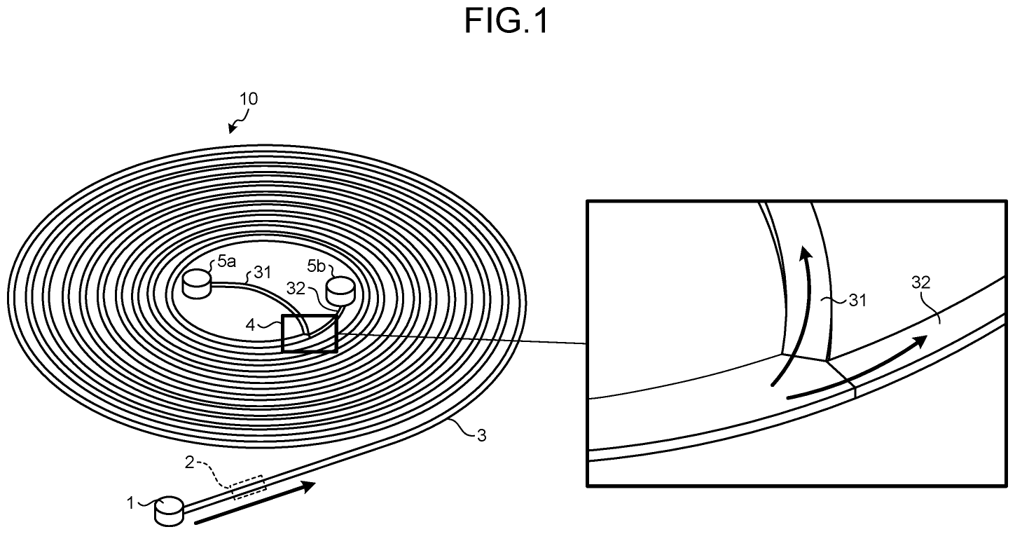

An example of a configuration of a microfluidic device according to a first embodiment will be described. FIG. 1 is a block diagram illustrating an example of the configuration of a microfluidic device 10 according to the first embodiment. As illustrated in FIG. 1, the microfluidic device 10 includes a fluid supply unit 1, an inflow part 2, a microchannel 3, a branch part 4, an inner collection unit 5a, and an outer collection unit 5b and classifies particles contained in a fluid. Specifically, the microfluidic device 10 separates a plurality of particles contained in a fluid according to sizes (particle sizes) of the particles.

The fluid supply unit 1 supplies the fluid containing the particles to the microchannel 3. Specifically, the fluid supply unit 1 supplies the fluid containing the particles having various sizes to the microchannel 3. For example, the fluid supply unit 1 supplies a fluid containing nucleated cells, which are subjected to the introduction of an initialization factor in establishment of iPS cells, to the microchannel 3. Here, the fluid containing nucleated cells may be blood (peripheral blood, cord blood, or other blood) containing nucleated cells, or a preservation solution containing nucleated cells in blood.

The fluid supply unit 1 is achieved by a syringe pump that is constituted of, for example, a syringe including a cylinder storing a fluid and a piston discharging the fluid stored in the cylinder from the tip of the cylinder, a connection unit connecting the tip of the cylinder and the microchannel 3, and a pump moving the piston. The fluid supply unit 1 is not limited to the syringe pump, and may also be achieved by a piston pump, a gear pump, a peristaltic pump, a piezoelectric micropump, or other pumps.

The fluid supply unit 1 injects the fluid into the microchannel 3 at a flow velocity that causes particles in the fluid to be separated according to sizes thereof in the microchannel 3. The speed of fluid (flow velocity of fluid) supplied by the fluid supply unit 1 can be changed according to the type of particles to be separated and other factors.

The inflow part 2 concentrates the particles contained in the fluid supplied by the fluid supply unit 1 in the vicinity of the center in the flow direction of the microchannel 3. For example, the inflow part 2 concentrates cells contained in blood in the vicinity of the center in the flow direction of the microchannel 3. The details of the inflow part 2 are described later. The inflow part 2 is also an example of an inflow part.

The microchannel 3 is formed in a helical shape and allows the fluid supplied by the fluid supply unit 1 to flow. Specifically, the microchannel 3 causes lift force and Dean vortex to act on the fluid flowing in the channel, thereby separating the particles contained in the fluid according to particle sizes. More specifically, the microchannel 3 separates the particles contained in the fluid at least in a first direction according to the sizes of the particles by the action of lift force, and separate the particles in a second direction by the action of flow (Dean vortex) in a channel cross-section. For example, the microchannel 3 receives an inflow of the fluid that contains cells having various sizes, separates the cells contained in the fluid in the first direction by the action of lift force, and separates the cells in the second direction by the action of flow in the channel cross-section. In one example, the microchannel 3 receives an inflow of a fluid that contains nucleated cells and non-nucleated cells contained in blood, separates the nucleated cells from the non-nucleated cells contained in the fluid in the first direction by the action of lift force, and separates the nucleated cells from the non-nucleated cells in the second direction by the action of flow in the channel cross-section. In other words, the microchannel 3 separates the nucleated cells from the non-nucleated cells contained in the blood by the action of lift force and Dean vortex in the channel. The microchannel 3 is an example of a microchannel.

The microchannel 3 is branched into a channel 31 and a channel 32 via the branch part 4. Specifically, the microchannel 3 is branched at the branch part 4 so that the particles separated according to the different particle sizes in the microchannel 3 flow into each of the channel 31 and the channel 32. For example, as illustrated in FIG. 1, the branch part 4 allows particles flowing inward in the flow direction to flow into the channel 31 and particles flowing outward in the flow direction to flow into the channel 32 in the microchannel 3 formed in a helical shape.

The channel 31 is a channel branched from the microchannel 3 via the branch part 4 and connected to the inner collection unit 5a. The channel 31 receives an inflow of the particles that have flowed inward in the flow direction in the microchannel 3 and discharges the particles into the inner collection unit 5a.

The channel 32 is a channel branched from the microchannel 3 via the branch part 4 and connected to the outer collection unit 5b. The channel 31 receives an inflow of the particles that have flowed outward in the flow direction in the microchannel 3 and discharges the particles into the outer collection unit 5b.

The inner collection unit 5a is connected to the channel 31 and collects particles that have flowed through the channel 31. The outer collection unit 5b is connected to the channel 32 and collects particles that have flowed through the channel 32.

The description of the configuration of the microfluidic device 10 according to the present embodiment has been given as described above. Under such a configuration, the microfluidic device 10 improves the classification performance. Specifically, the microfluidic device 10 improves the classification performance in the microchannel where the particles contained in the fluid are classified by the action of lift force and Dean vortex.

The microchannel where particles contained in the fluid are classified by the action of lift force and Dean vortex allows relatively large particles in the fluid to move closer to upper and lower walls of the microchannel by the action of lift force, and allows particles near the upper and lower walls to move inward in the microchannel and particles in the vicinity of the center of the channel to move outward in the microchannel by the action of Dean vortex. Therefore, the particles contained in the fluid are concentrated and separated by particle size.

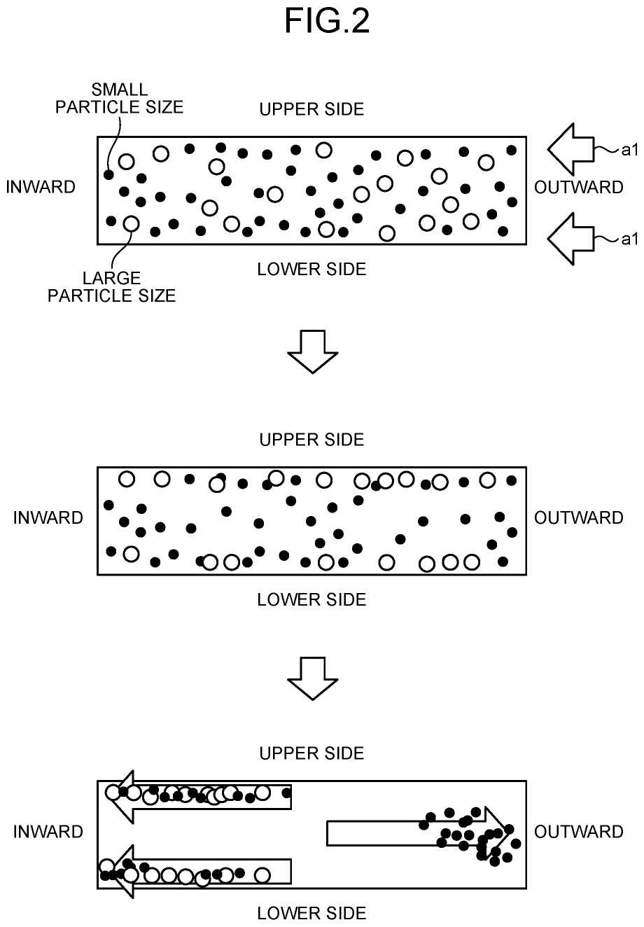

However, in the microchannel where the particles contained in the fluid are classified by the action of lift force and Dean vortex, the particles may not be sufficiently separated during the separation by the action of lift force. FIG. 2 is a diagram for explaining a microchannel that classifies particles contained in a fluid by the action of lift force and Dean vortex. Here, a cross-sectional view of the microchannel in a direction perpendicular to the flow direction of the fluid is illustrated in FIG. 2. The rectangle in FIG. 2 illustrates the inner wall of the microchannel, and the black and white circles within the rectangle illustrate a distribution state of particles in the microchannel. In FIG. 2, the upper figure illustrates a distribution state of particles when a fluid containing large size particles and small size particles is injected into the microchannel, the middle figure illustrates a distribution state of the particles when the particles are moved by the action of lift force, and the lower figure illustrates a distribution state of the particles when the particles are moved by the action of Dean vortex.

As illustrated in the upper figure in FIG. 2, in a case in which the fluid is injected into the microchannel, the large size particles and the small size particles are dispersed and distributed in the channel. In other words, as illustrated by arrow al in the upper figure in FIG. 2, the large size particles and the small size particles are distributed near the upper (vertical upper side) and lower (vertical lower side) walls in the microchannel.

In a case in which lift force acts under such a distribution state of the particles, as illustrated in the middle figure in FIG. 2, the large size particles are moved to near the upper and lower walls, but the small size particles that have existed near the upper and lower walls beforehand also stay near the upper and lower walls. In other words, the large size particles and the small size particles are present in a mixed state near the upper limit wall of the microchannel after the action of lift force.

In a case in which Dean vortex further acts in such a distribution state of the particles, as illustrated in the lower figure in FIG. 2, a particle group consisting a mixture of the large size particles and the small size particles is concentrated inward in the flow direction of the fluid in the helical microchannel, and a particle group consisting of the small size particles is concentrated outward.

Thus, in the microchannel that classifies the particles contained in the fluid by the action of lift force and Dean vortex, in a case in which the small size particles are present near the upper and lower walls beforehand, the large size particles are not sufficiently separated from the small size particles, and a group in which both size particles are mixed is collected.

Therefore, the microfluidic device 10 according to the present embodiment allows the fluid containing the particles to flow into the vicinity of the center of the cross-section perpendicular to the flow direction of the fluid, in an area where lift force acts in the microchannel 3 and separates the particles contained in the fluid according to the sizes of the particles by the action of lift force. Specifically, the microfluidic device 10 is configured to concentrate the particles contained in the fluid in the vicinity of the center in the channel of the microchannel, and then to cause the lift force and Dean vortex to act, thereby improving the classification performance of the particles contained in the fluid. More specifically, the microfluidic device 10 concentrates, by the inflow part 2, the fluid supplied from the fluid supply unit 1 into the vicinity of the center in the channel of the microchannel 3, thereby concentrating the small size particles that have existed beforehand near the upper and lower walls, and allows only large size particles to be present on the upper limit wall after the action of lift force, thereby improving the classification performance of the particles contained in the fluid.

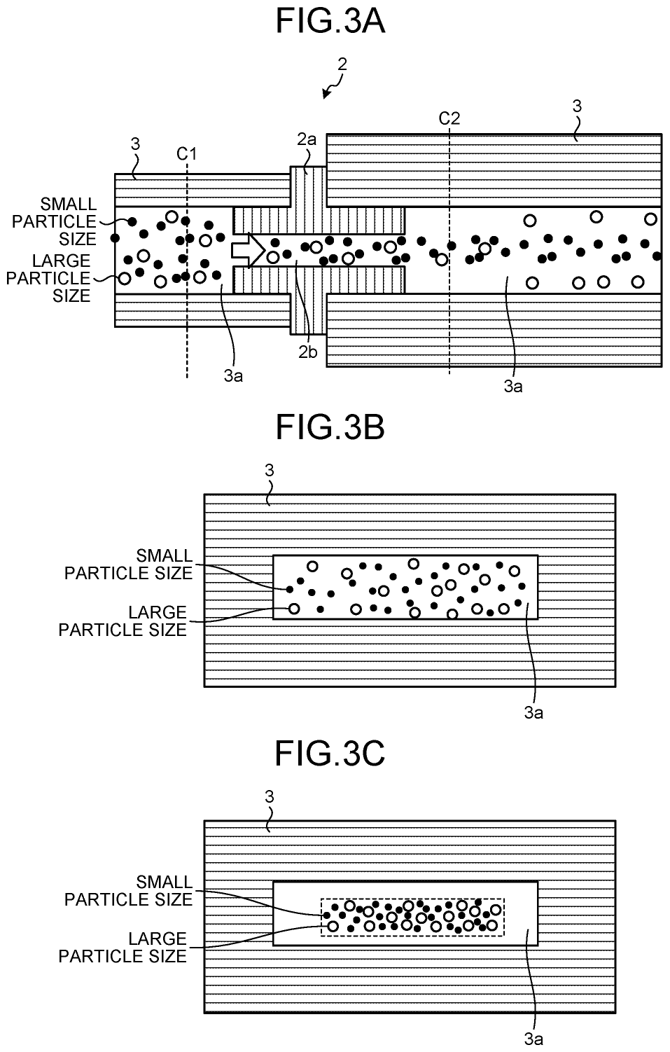

A detailed description of the inflow part 2 according to the present embodiment will be described below. FIG. 3A to FIG. 3C are diagrams illustrating an example of the inflow part 2 according to the first embodiment. Here, FIG. 3A is a cross-sectional view along the flow direction of the fluid in the inflow part 2 and the microchannel 3. FIG. 3B illustrates a cross-section C1 in FIG. 3A, and FIG. 3C illustrates a cross-section C2 in FIG. 3A.

The inflow part 2 is provided on the upstream of the area where lift force acts in the microchannel 3 and allows the fluid to flow into the area where lift force acts. Here, a length of a channel cross-section of the inflow part 2 in the first direction is formed to be smaller than a length of a channel cross-section of the area where lift force acts in the first direction. In addition, a length of a channel cross-section of the inflow part 2 in the second direction is formed to be smaller than a length of a channel cross-section of the area where lift force acts in the second direction. In other words, the inflow part 2 has a smaller cross-sectional area perpendicular to the flow direction of the fluid than a cross-sectional area in the area where lift force acts.

For example, as illustrated in FIG. 3A, the inflow part 2 is achieved by an inlet 2a disposed between channels on the upstream from the area where lift force acts in the microchannel 3. Here, the inlet 2a is provided to allow the fluid to flow in the same direction as the flow direction of the fluid in the area where lift force acts.

In other words, the inlet 2a is provided so that an orientation of a channel interior 2b through which the fluid flows via the inlet 2a is matched with an orientation of a channel interior 3a through which the fluid flows at the downstream of the inlet 2a in the microchannel 3. As a result, the particles contained in the fluid passing through the inlet 2a flow into the vicinity of the center of the microchannel 3 without heading toward near the walls of the microchannel 3.

For example, even though the particles (large size particles and small size particles) contained in the fluid supplied to the microchannel 3 by the fluid supply unit 1 are dispersed and distributed throughout the entire area of the channel interior 3a, as illustrated in FIG. 3B, the particles pass through the inlet 2a to be concentrated in the vicinity of the center of the channel interior 3a as illustrated in FIG. 3C. As a result, the small size particles are removed from near the upper and lower walls of the channel interior 3a, and only large size particles are present near the upper and lower walls of the channel interior 3a after the action of lift force.

By Dean vortex acting in this state, the microfluidic device 10 can allow the particle group consisting of the large size particles to be concentrated inward in the flow direction of the fluid in the helical microchannel 3 and allow the particle group consisting of the small size particles to be concentrated outward. In other words, the microchannel 3 has a helical partial channel, and in the helical partial channel, the large size particles congregate inward in a helix and the small size particles congregate outward in the helix by the action of flow in the channel cross-section.

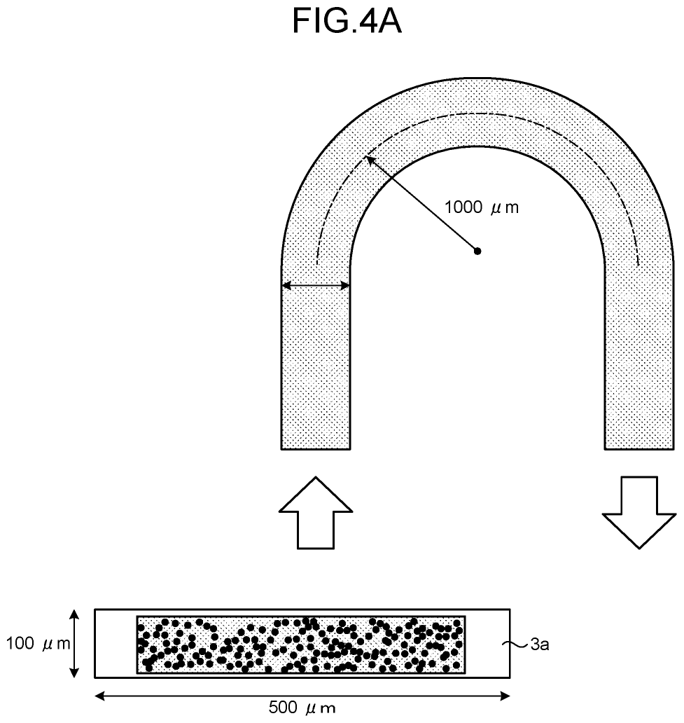

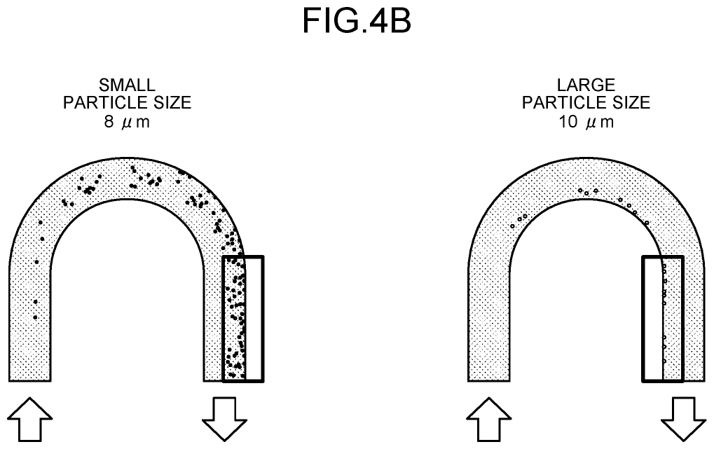

The result of simulating the classification method described above is illustrated using FIGS. 4A and 4B below. FIGS. 4A and 4B are diagrams for explaining the result of the simulation according to the first embodiment. In this simulation, as illustrated in FIG. 4A, a case of a fluid containing particles (cells) flowing in a microchannel in which the channel interior 3a is formed with “height: 100 μm, width: 500 μm” and an arc with “radius: 1000 μm” will be described.

As illustrated in FIG. 4A, in a case in which the fluid is allowed to flow in the channel interior of the microchannel so that the cells flow in the center of the microchannel in the cross-section described above, as illustrated in FIG. 4B, a result in which particles having a small size of “particle size: 8 μm” are concentrated on an outward wall in the microchannel and particles having a large size of “particle size: 10 μm” are concentrated on an inward wall in the microchannel was obtained. This result indicates that the classification method described above can accurately separate nucleated cells (particle size: about 7 to 12 μm) from non-nucleated cells (about 6 to 8 μm) in blood.

Here, the inflow part 2 is desirably formed such that the cross-sectional area thereof on the side where the fluid flows into the area where lift force acts is equal to or less than 90% of the cross-sectional area of the area where lift force acts. For example, a cross-sectional area of the channel interior 2b in the inlet 2a illustrated in FIG. 3A along the vertical direction is desirably equal to or less than 90% of an area of the cross-section C2 of the channel interior 3a.

As described above, according to the first embodiment, the microchannel 3 separates the particles contained in the fluid according to the sizes of the particles by the action of lift force. The inflow part 2 is provided on the upstream of the area where lift force acts in the microchannel 3, and the cross-sectional area perpendicular to the flow direction of the fluid is smaller than the cross-sectional area in the area where lift force acts, thereby allowing the fluid to flow into the area where lift force acts. Therefore, the microfluidic device 10 according to the first embodiment can remove the small size particles from near the upper and lower walls in the area where lift force acts, and can prevent particles having various sizes from being mixed in the particle group after the action of lift force to improve the classification performance.

According to the first embodiment, the inflow part 2 is provided to allow the fluid to flow in the same direction as the flow direction of the fluid in the area where lift force acts. Therefore, the microfluidic device 10 according to the first embodiment can prevent the particles from flowing toward the walls, thereby further improving the classification performance.

According to the first embodiment, the inflow part 2 is formed such that the cross-sectional area thereof on the side where the fluid flows into the area where lift force acts is equal to or less than 90% of the cross-sectional area of the area where lift force acts. Thus, the microfluidic device 10 according to the first embodiment may allow the particles to more reliably concentrate toward the center inside the channel of the microchannel.

### Other Embodiments

The first embodiment has been described so far, but various embodiments may be implemented in different forms other than the first embodiment described above.

In the above-described embodiment, the case in which the channel interior of the inflow part 2 is shorter than the channel interior of the microchannel 3 in both distances in the vertical direction (height direction) and in the inward and outward direction (width direction) has been described. However, the embodiment is not limited thereto, and the distance in the height direction of the channel interior of the inflow part 2 may be shorter than the channel interior of the microchannel 3. In other words, the distance in the height direction in the cross-section of the inflow part 2 is shorter than the cross-section in the area where lift force acts.

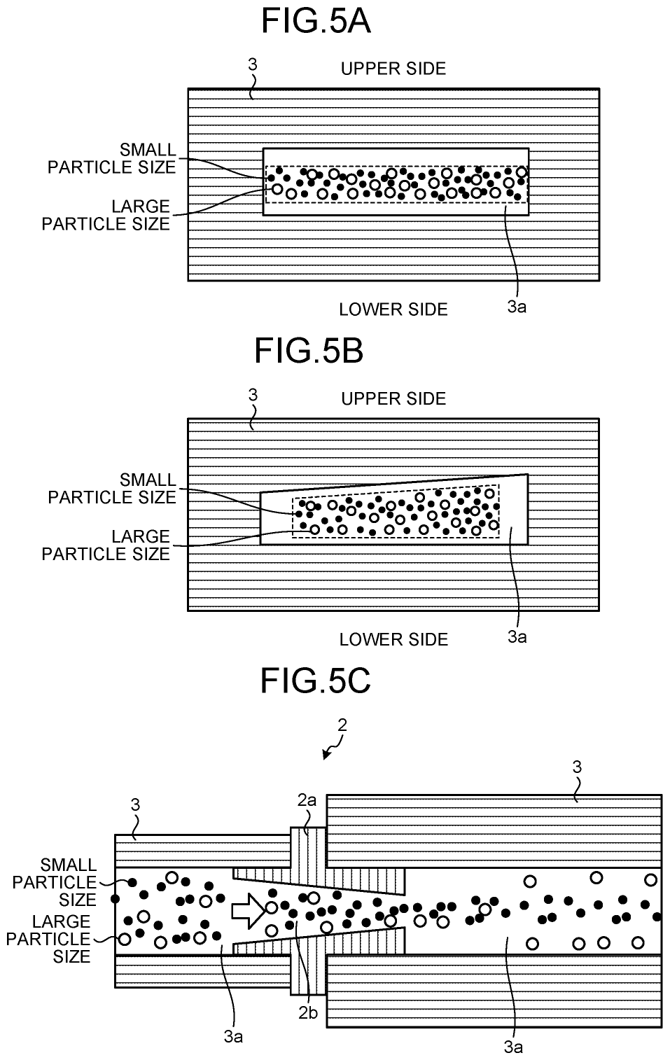

FIG. 5A is a diagram for explaining an example of the inflow part 2 according to another embodiment. For example, the distance in the width direction of the inflow part 2 may be formed in the same distance in the width direction of the channel interior 3a of the microchannel 3 in the cross-section perpendicular to the flow direction of the fluid, as illustrated in FIG. 5A, and the distance in the height direction may be shorter than a distance in the height direction of the channel interior 3a of the microchannel 3. Therefore, the microfluidic device 10 can reduce a difference between a size of the channel interior in the inflow part 2 and a size of the channel interior in the microchannel 3, thereby enabling control for the flow of the fluid to be easier.

In the embodiment described above, the case in which a cross-sectional shape of the channel interior in the microchannel 3 is rectangular has been described. However, the embodiment is not limited thereto, and the channel interior may be formed in any shape. In such cases, the inflow part 2 is formed so that the cross-sectional shape of the channel interior is formed in the same shape as the cross-sectional shape of the area where lift force acts.

FIG. 5B is a diagram for explaining an example of shapes of the inflow part 2 and the microchannel 3 according to the other embodiment. For example, as illustrated in FIG. 5B, the microchannel 3 may be formed in a trapezoidal shape in which the outward distance in the cross-sectional shape of the channel interior 3a is longer than the inward distance.

In this case, the inflow part 2 is formed such that the cross-sectional shape of the channel interior is the same as that of the microchannel 3. In other words, as illustrated in FIG. 5B, the inflow part 2 is also formed in a trapezoidal shape in which the outward distance in the cross-sectional shape of the channel interior is longer than the inward distance. As a result, the microfluidic device 10 can allow the lift force uniformly to act on the particles contained in the fluid, and can prevent degradation of the classification performance.

In the embodiment described above, the case in which the size of the channel interior in the inflow part 2 is uniform along the flow direction of the fluid has been described. However, the embodiment is not limited thereto, and the size of the channel interior in the inflow part 2 may vary along the flow direction of the fluid.

FIG. 5C is a diagram illustrating an example of a shape of the inflow part 2 according to the other embodiment. For example, as illustrated in FIG. 5C, the inflow part 2 is formed such that a cross-sectional area on the side where the fluid flows is larger than the cross-sectional area on the side where the fluid flows into the area where lift force acts. Therefore, the microfluidic device 10 can allow the fluid to flow smoothly into the inflow part 2.

In the embodiment described above, the case in which one channel is provided in the inflow part 2 has been described. However, the embodiment is not limited thereto, and a plurality of the channels may also be formed in the inflow part. In such a case, the inflow part 2 is formed with the channels, which are arranged in the vertical direction, and allows the fluid to flow from each of the channels to the area where lift force acts.

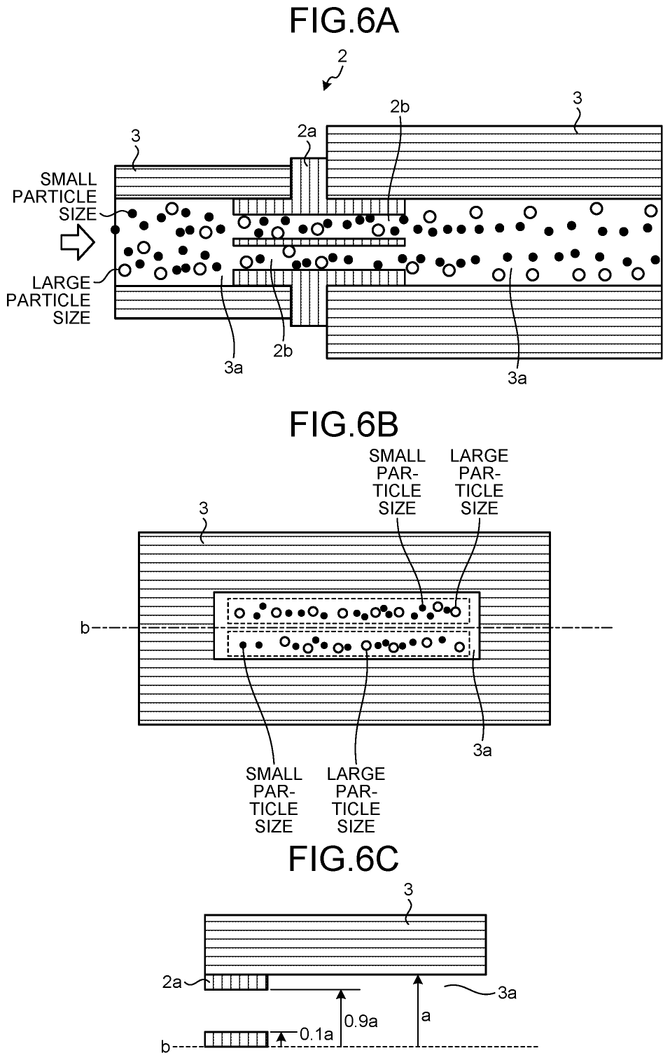

FIGS. 6A to 6C are diagrams illustrating an example of a shape of the inflow part 2 according to another embodiment. For example, the inflow part 2 has two channels (channel interior 2b) aligned vertically, as illustrated in FIG. 6A. Here, the two channels are formed not to include the center of the channel interior 3a of the microchannel 3 in the height direction.

In other words, as illustrated in FIG. 6B, the inflow part 2 includes upper and lower channels each of which receives an inflow of the fluid using a center b of the channel interior 3a of the microchannel 3 in the height direction as a boundary. Here, in a case in which the two channels are formed in the inflow part 2, the channels are formed in the inflow part 2 within a range of “0.1 to 0.9” in the height direction, which is calculated from the center b of the area where lift force acts in the microchannel 3 in the height direction.

For example, the channel formed on the upper side using the center “b” as a boundary in the height direction is formed at a position extending over “0.1a” to “0.9a” of a distance “a” from the center “b” to the upper inward wall, as illustrated in FIG. 6C. Therefore, the microfluidic device can control the particles not to present at a position of the center b in the height direction where lift force does not act, and enables the classification performance to be further improved.

In the embodiment described above, the case in which the inflow part 2 is achieved by the inlet 2a disposed between the channels of the microchannel 3 has also been described. However, the embodiment is not limited thereto, and the inflow part 2 may be formed inside the channel interior of the microchannel 3. For example, a case in which an inflow part 2 that narrows a channel toward the inward wall is formed at the upstream of the area where lift force acts in the microchannel 3 may be adopted.

In the embodiment described above, the case in which the inflow part 2 is achieved by the inlet 2a disposed between the channels of the microchannel 3 has also been described. However, the embodiment is not limited thereto, and the inflow part 2 may be achieved by a microchannel 3 with a channel interior having various cross-sectional areas through which the fluid flows.

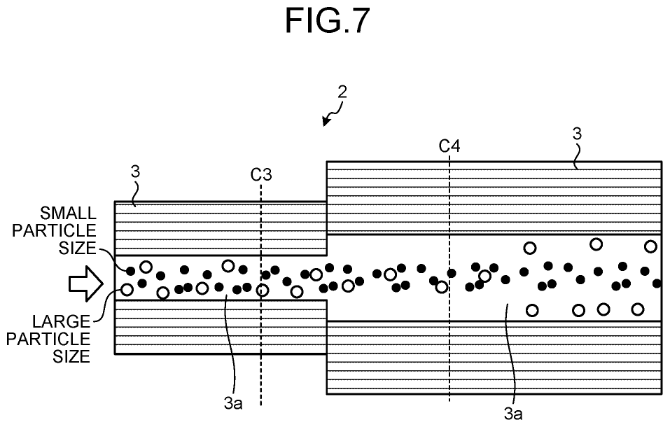

FIG. 7 is a diagram illustrating an example of a shape of an inflow part according to still yet another embodiment. For example, the inflow part 2 is formed by a microchannel 3 with a channel interior having various cross-sectional areas through which the fluid flows, as illustrated in FIG. 7. Here, the microchannel 3 is formed such that an area of a channel interior in a cross-section C3 at the upstream of the area where lift force acts is smaller than an area of a channel interior in a cross-section C4 in the area where lift force acts, as illustrated in FIG. 7. Therefore, the microfluidic device 10 can allow the particles to flow into the area where lift force acts in a state in which the particles congregate in the vicinity of the center of the channel, and enables the classification performance to be improved.

In the embodiments described above, blood cells are described as one example of the particles, but the embodiments are not limited thereto, and various other particles can be intended.

According to at least one of the embodiments described above, the classification performance can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A microfluidic device comprising:
a microchannel configured to separate particles contained in a fluid at least in a first direction according to a size of the particles by an action of lift force and separate the particles in a second direction by an action of flow in a channel cross-section; and
an inflow part configured to allow the fluid to flow into an area where the lift force acts, the inflow part being provided at an upstream from the area where the lift force acts in the microchannel, wherein
a length of a channel cross-section of the inflow part in the first direction is formed to be smaller than a length of a channel cross-section of the area where the lift force acts in the first direction.

2. The microfluidic device according to claim 1, wherein the inflow part is provided to allow the fluid to flow in the same direction as a flow direction of the fluid in the area where the lift force acts.

3. The microfluidic device according to claim 1, wherein a length of a channel cross-section of the inflow part in the second direction is formed to be smaller than a length of a channel cross-section of the area where the lift force acts in the second direction.

4. The microfluidic device according to claim 3, wherein a shape of the channel cross-section in the inflow part is formed to be a same shape as a shape of the channel cross-section in the area where the lift force acts.

5. The microfluidic device according to claim 1, wherein the inflow part is formed such that a channel cross-sectional area on a side where the fluid flows is larger than a channel cross-sectional area on a side where the fluid flows into the area where the lift force acts.

6. The microfluidic device according to claim 1, wherein the inflow part is formed such that the channel cross-sectional area on the side where the fluid flows into the area where the lift force acts is equal to or less than 90% of the channel cross-sectional area in the area where the lift force acts.

7. The microfluidic device according to claim 1, wherein the inflow part is formed with a plurality of channels, which are arranged in a vertical direction, and allows the fluid to flow from each of the channels into the area where the lift force acts.

8. The microfluidic device according to claim 7, wherein the channels are formed in the inflow part within a range of 0.1 to 0.9 in a height direction, which is calculated from a center of the area where the lift force acts in the microchannel in the height direction.

9. The microfluidic device according to claim 1, wherein
the microchannel includes a helical partial channel, and
in the helical partial channel, large size particles congregate inward in a helix, and small size particles congregate outward in the helix by the action of the flow in the channel cross-section.

10. The microfluidic device according to claim 9, wherein the microchannel separates the particles such that the large size particles are brought closer to a channel wall by the action of the lift force.

11. The microfluidic device according to claim 1, wherein the microchannel receives an inflow of a fluid that contains cells having various sizes, separates the cells contained in the fluid in the first direction by the action of the lift force, and separates the cells in the second direction by the action of the flow in the channel cross-section.

12. The microfluidic device according to claim 1, wherein the microchannel receives an inflow of a fluid that contains nucleated cells and non-nucleated cells contained in blood, separates the nucleated cells from the non-nucleated cells contained in the fluid in the first direction by the action of the lift force, and separates the nucleated cells from the non-nucleated cells in the second direction by the action of the flow in the channel cross-section.

13. A classification method comprising:
allowing a fluid containing particles to flow into a vicinity of a center of an area where lift force acts in a microchannel in a cross-section perpendicular to a flow direction of the fluid; and
separating the particles contained in the fluid according to a size of the particles by an action of the lift force.

14. A classification method of separating particles contained in a fluid in at least a first direction according to a size of the particles by an action of lift force and separating the particles in a second direction by an action of flow in a channel cross-section, the method comprising allowing the fluid to flow into an area where the lift force acts from an inflow part configured to allow the fluid to flow into the area where the lift force acts, in which a length of a channel cross-section of the inflow part in the first direction is formed to be smaller than a length of a channel cross-section of the area where the lift force acts in the first direction.
